(19) United States
(12) Patent Application Publication
(10) Pub. No.: US 2026/0363907 A1
(43) Pub. Date: Nov. 19, 2026

(54) DELIVERY OF VISCOELASTIC AND OCULAR IMPLANT

(71) Applicant: Alcon Inc., Fribourg (CH)

(72) Inventors: Wayne Noda, Mission Viejo, CA (US); Daniel Hyman, Foothill Ranch, CA (US); David Kimball, Ammon, ID (US); Jestwin Edwin Lee, IV, Grandview, TX (US); Ahmad Robert Hadba, Fort Worth, TX (US)

(21) Appl. No.: 19/676,701

(22) Filed: May 13, 2026

Related U.S. Application Data
(60) Provisional application No. 63/805,722, filed on May 14, 2025.

(51) Int. Cl. A61F 9/00; A61F 9/007

(52) U.S. Cl. CPC A61F 9/0017; A61F 9/00781

(57) ABSTRACT

An apparatus for delivering viscoelastic material and an implant to an eye. The apparatus may generally comprise a cannula having a conduit chamber and an implant chamber, a conduit rack aligned with the conduit chamber, a conduit coupled to the conduit rack, an implant rack aligned with the implant chamber, a control rod coupled to the implant rack and configured to be coupled to the implant in the implant chamber, and a controller configured to selectively move the conduit rack or the implant rack. The conduit may be configured to move through the conduit chamber and to be coupled to a source of the viscoelastic material.

### PRIORITY CLAIM

This application claims the benefit of priority of U.S. Provisional Patent Application Serial No. 63/805,722 entitled, “DELIVERY OF VISCOELASTIC AND OCULAR IMPLANT” filed on May 14, 2025, whose inventors are Wayne Noda, Daniel Hyman, David Kimball, Jestwin Edwin Lee IV and Ahmad Robert Hadba, which is hereby incorporated by reference in its entirety as though fully and completely set forth herein.

### TECHNICAL FIELD

The invention set forth in the appended claims relates generally to ophthalmic surgery and treatment, including, without limitation, delivery of viscoelastic material and an ocular implant.

### BACKGROUND

The human eye can suffer a number of maladies causing mild deterioration to complete loss of vision. For example, glaucoma is a condition of increased pressure in an eye, and researchers have theorized that prolonged exposure to high intraocular pressure can damage the optic nerve that transmits sensory information from the eye to the brain. This damage to the optic nerve can result in loss of peripheral vision. As glaucoma progresses, more and more of the visual field is lost until vision is completely lost. For this reason, eye care professionals routinely screen patients for glaucoma by measuring intraocular pressure.

It is commonly believed that pressure inside the eye can begin to rise if natural drainage systems in the eye stop functioning properly. Thus, treatments for glaucoma often focus on relieving the pressure. Canaloplasty is one surgical technique that can promote outflow of aqueous via the natural drainage systems through the trabecular meshwork and into Schlemm’s canal. Certain types of devices can also be implanted into Schlemm’s canal to promote drainage and maintain proper pressure in the eye.

While the benefits of canaloplasty and ocular implants for treating glaucoma are known, improvements to delivery systems, components, and processes continue to improve outcomes and benefit patients.

### BRIEF SUMMARY

New and useful systems, apparatuses, and methods for surgical delivery of viscoelastic fluid and ocular implants to an eye are set forth in the appended claims. Illustrative embodiments are also provided to enable a person skilled in the art to make and use the claimed subject matter.

For example, some embodiments comprise an apparatus for delivering viscoelastic material and an implant to an eye. In some aspects, the apparatus may comprise a cannula having a conduit chamber and an implant chamber, a conduit rack aligned with the conduit chamber, a conduit coupled to the conduit rack, an implant rack aligned with the implant chamber, a control rod coupled to the implant rack and configured to be coupled to the implant in the implant chamber, and a controller configured to selectively move the conduit rack or the implant rack. The conduit may be configured to move through the conduit chamber and to be coupled to a source of the viscoelastic material.

In more particular embodiments, the apparatus may comprise a pinion coupled to the controller, and a switch operable to move the pinion from a first position to a second position. The pinion may be coupled to the conduit rack in the first position and coupled to the implant rack in the second position. Moving the controller can cause the pinion to move the conduit rack relative to the conduit chamber if the pinion is in the first position and can cause the pinion to move the implant rack relative to the implant chamber if the pinion is in the second position. The pinion may be configured to remain coupled to the controller as the pinion is moved from the first position to the second position.

In some embodiments, the apparatus may further comprise a flow controller coupled to the conduit and configured to couple the conduit to the source of viscoelastic material. An actuator may be configured to operate the flow controller to deliver viscoelastic material through the conduit to the eye.

In some embodiments, an apparatus for delivering viscoelastic material and an implant to an eye may comprise a conduit cannula and an implant cannula. A first slider may be configured to extend and retract the conduit cannula, and a second slider may be configured to extend and retract the implant cannula. A conduit rack may be aligned with the conduit cannula, and a conduit may be coupled to the conduit rack. The conduit can be configured to move through the conduit cannula and to be coupled to a source of the viscoelastic material. An implant rack may be aligned with the implant cannula. A control rod can be coupled to the implant rack and configured to be coupled to the implant. A controller can be configured to selectively move the conduit rack or the implant rack.

In some embodiments an apparatus for eye surgery may comprise a cannula comprising a conduit chamber and an implant chamber, a conduit configured to move through the conduit chamber, a control rod configured to be coupled to an implant in the implant chamber, a flow controller coupled to the conduit, a source of viscoelastic material coupled to the flow controller, a controller configured to selectively extend and retract the conduit and the control rod, and an actuator configured to operate the flow controller. The controller may be operable to selectively position the conduit through the conduit chamber into the eye and deliver the implant through the implant chamber into the eye, and the actuator can be configured to deliver viscoelastic material through the conduit to the eye.

The systems, apparatuses, and methods described here may be particularly advantageous for treating glaucoma with canaloplasty and ocular implants.

Features, elements, and aspects described in the context of some embodiments may also be omitted, combined, or replaced by alternative features. Other features, objectives, advantages, and a preferred mode of making and using the claimed subject matter are described in greater detail below with reference to the accompanying drawings of illustrative embodiments.

### BRIEF DESCRIPTION OF THE DRAWINGS

The accompanying drawings illustrate some objectives, advantages, and a preferred mode of making and using some embodiments of the claimed subject matter. Like reference numbers represent like parts in the examples.

FIG. 1 is a schematic view of an interior of a human eye.

FIG. 2 is a schematic representation of an apparatus being used by a physician to treat the eye of a patient.

FIG. 3A is a schematic view of an example of a cannula that may be associated with the apparatus of FIG. 2.

FIG. 3B is perspective view of a distal end of the cannula of FIG. 3A.

FIG. 3C is another perspective view of a distal end of the cannula of FIG. 3A.

FIG. 4 is a schematic view of another example of the apparatus of FIG. 2.

FIG. 5 is a perspective view of a portion of the apparatus of FIG. 4.

FIG. 6 is a schematic side view of a flow controller that may be associated with the apparatus of FIG. 5.

FIGS. 7A-7C are schematic views of another example of a cannula in different configurations that may be associated with the apparatus of FIG. 2.

FIGS. 8A-8B are schematic views of another example of the cannula that may be associated with the apparatus of FIG. 2.

FIG. 9 is a perspective view of another example of a flow controller.

FIG. 10 is another view of the flow controller of FIG. 9.

FIG. 11 is a section view of the flow controller of FIG. 9.

FIG. 12 is a schematic view of another example of the flow controller.

FIG. 13 is a perspective view of another example of the flow controller.

FIGS. 14A-14C are schematic diagrams of examples of an actuator that may be associated with the apparatus of FIG. 2.

FIG. 15 is a side view of another example of the flow controller.

FIG. 16 is a schematic section view of another example of the flow controller.

FIG. 17 is a side view of a piston that may be associated with the flow controller of FIG. 16.

FIG. 18 is a schematic view of the flow controller of FIG. 16 in a second configuration.

FIG. 19 is a schematic section view of another example of the flow controller.

FIG. 20 is a schematic section view of the flow controller of FIG. 19 in a second configuration.

FIGS. 21A and 21B are side views of an example of a visco module that may be associated with some examples of the apparatus.

FIG. 22 is a transparent view of the visco module of FIG. 21B.

FIG. 23 is a section view of the visco module of FIG. 22.

FIG. 24 is a perspective view of another example of the apparatus of FIG. 2.

FIGS. 25A-25B and 26A-26B illustrate various embodiments of a protective cover that may be associated with the apparatus of FIG. 24.

FIG. 27 is a perspective view of another example of the apparatus of FIG. 2.

FIG. 28 is a top view of the apparatus 200 of FIG. 27.

### DESCRIPTION OF EXAMPLE EMBODIMENTS

The following description of example embodiments provides information that enables a person skilled in the art to make and use the subject matter set forth in the appended claims, but it may omit certain details already well known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient receiving treatment. However, as should be recognized by those skilled in the art, this frame of reference is merely a descriptive expedient rather than a strict prescription.

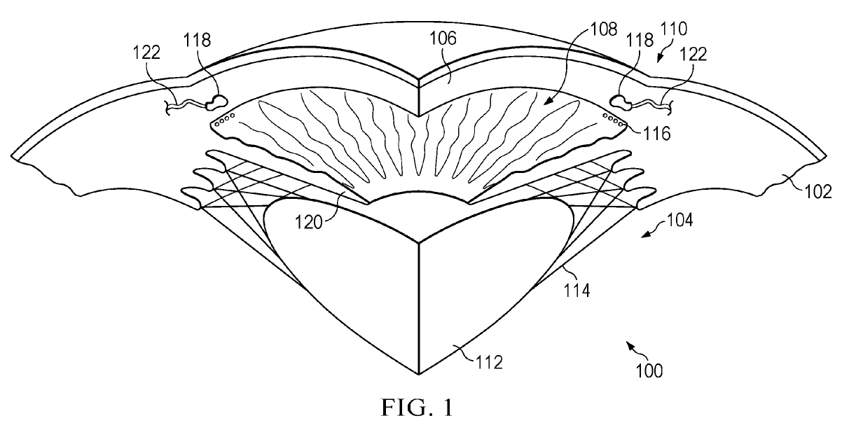

FIG. 1 is a schematic view of an interior of a human eye 100. Eye 100 can be conceptualized as a fluid filled ball having two chambers. Sclera 102 of eye 100 surrounds a posterior chamber 104 filled with a viscous fluid known as vitreous humor. Cornea 106 of eye 100 encloses an anterior chamber 108 that is filled with a fluid known as aqueous humor. The cornea 106 meets sclera 102 at a limbus 110 of eye 100. A lens 112 of eye 100 is located between anterior chamber 108 and posterior chamber 104. Lens 112 is held in place by a number of ciliary zonules 114. Whenever a person views an object, that object is viewed through the cornea, the aqueous humor, and the lens of the eye. To be transparent, the cornea and the lens can include no blood vessels. Accordingly, no blood flows through the cornea and the lens to provide nutrition to these tissues and to remove wastes from these tissues. Instead, these functions are performed by the aqueous humor. A continuous flow of aqueous humor through the eye provides nutrition to portions of the eye (e.g., the cornea and the lens) that have no blood vessels. This flow of aqueous humor also removes waste from these tissues.

Aqueous humor is produced by an organ known as the ciliary body. The ciliary body includes epithelial cells that continuously secrete aqueous humor. In a healthy eye, a stream of aqueous humor flows out of the eye as new aqueous humor is secreted by the epithelial cells of the ciliary body. This excess aqueous humor enters the blood stream and is carried away by venous blood leaving the eye.

In a healthy eye, aqueous humor flows out of the anterior chamber 108 through the trabecular meshwork 116 and into Schlemm’s canal 118, located at the outer edge of the iris 120. Aqueous humor exits Schlemm’s canal 118 by flowing through a number of outlets 122. After leaving Schlemm’s canal 118, aqueous humor is absorbed into the venous blood stream.

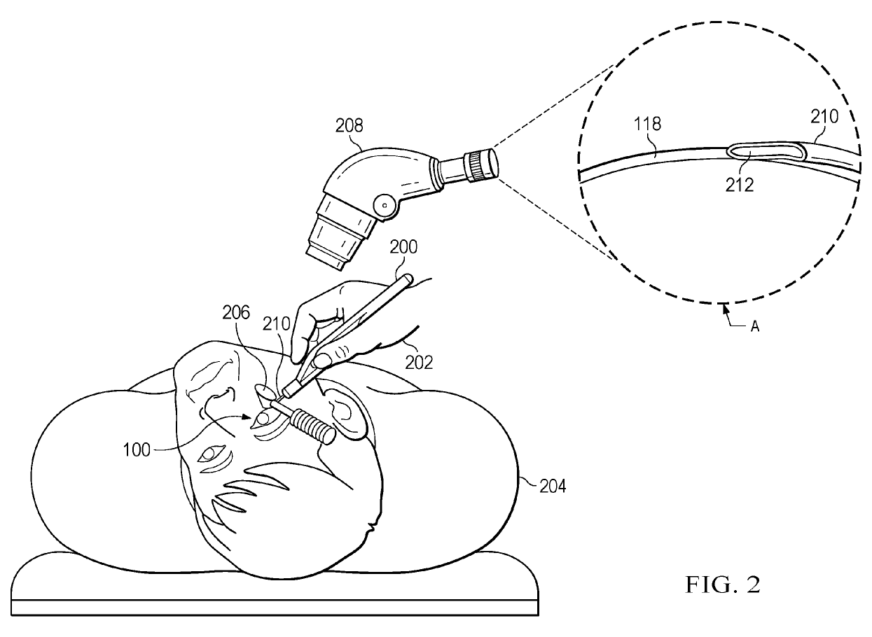

FIG. 2 is a schematic representation of an apparatus 200 being used by a physician 202 to treat the eye 100 of a patient 204. In the example procedure of FIG. 2, the apparatus 200 is held in the right hand of the physician 202. The left hand (not shown) may be used to hold the handle of a gonio lens 206. Alternatively, some physicians may prefer holding the apparatus 200 in the left hand and the gonio lens 206 in the right hand.

During the example procedure of FIG. 2, the physician 202 may view the interior of the eye 100 using the gonio lens 206 and a microscope 208. Detail A of FIG. 2 is a stylized representation of an image that may be viewed by the physician 202 through the microscope 208. For example, a distal portion of a cannula 210 of the apparatus 200 is visible in Detail A of FIG. 2, as well as the location of Schlemm’s canal 118, which is lying under various tissues (e.g., the trabecular meshwork) that surround the anterior chamber. For some procedures, such as a canaloplasty, a distal opening 212 of cannula 210 can be positioned near Schlemm’s canal 118 of the eye 100.

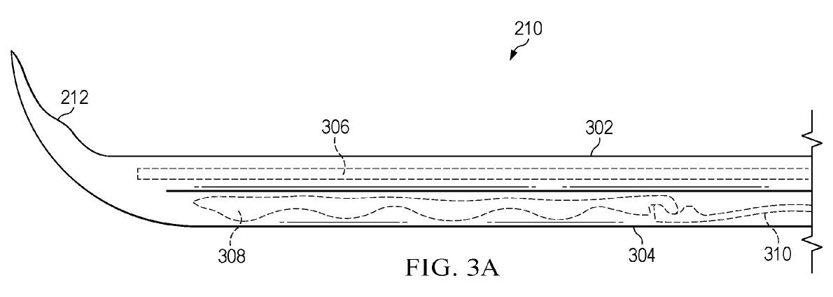

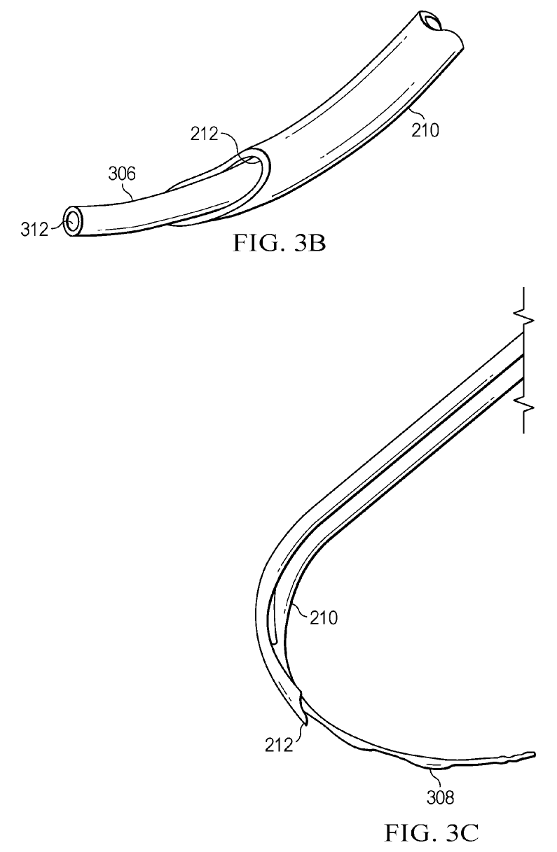

FIG. 3A is a schematic section view of one embodiment of the cannula 210 configured to deliver viscoelastic material and an ocular implant. In the example of FIG. 3A, the cannula 210 comprises a conduit chamber 302 and an implant chamber 304. The conduit chamber 302 and the implant chamber 304 of FIG. 3A are disposed adjacent to and parallel to each other within the cannula 210.

In some embodiments, a conduit 306 may be disposed in the conduit chamber 302. A “conduit,” in this context, broadly includes a tube, pipe, hose, or other structure with one or more lumina or open pathways adapted to convey a fluid or viscoelastic material between two ends. Typically, a conduit is an elongated, cylindrical structure with some flexibility, but the geometry and rigidity may vary. Moreover, some conduits may be molded into or otherwise integrally combined with other components. A microcatheter may be a suitable conduit for some embodiments. In some embodiments, the conduit 306 may be made of a polyethylene, polyurethane, polyamide, or other suitable material. For example, Grilamid® or VESTAMID® polyamide or Pebax® elastomer may be suitable for some embodiments. In some embodiments, the conduit 306 can have a size and a cross-sectional shape that matches the size and cross-sectional shape of the interior of the conduit chamber 302. For example, an outside diameter of 0.008 inch and inside diameter of 0.006 inch may be suitable for some embodiments. Suitable cross-sections may be circular or oval, for example. More generally, the conduit 306 can be any shape so long as it is able to be slidably disposed within the conduit chamber 302 and the cannula 210.

In some embodiments, the conduit 306 can be configured to be advanced out of the cannula 210 into Schlemm’s canal. In some procedures, such as a canaloplasty, the conduit 306 can be advanced around Schlemm’s canal, which can dilate Schlemm’s canal to promote outflow of aqueous via the natural outflow pathways through the trabecular meshwork. In some embodiments, a viscoelastic material, also commonly known as an ophthalmic viscosurgical device (OVD), can also be administered through the conduit 306 into Schlemm’s canal to further dilate Schlemm’s canal. Additionally, or alternatively, an ocular implant may be advanced through the cannula 210 into Schlemm’s canal. The viscoelastic material can be delivered into Schlemm's canal before or after delivering an ocular implant.

FIG. 3A illustrates an example of an implant 308 may be disposed in the implant chamber 304. In some embodiments, the implant 308 may be a microstent made of a flexible and biocompatible material, such as nitinol. Some embodiments of the implant 308 may be shaped to match the curvature of Schlemm’s canal. In the example of FIG. 3A, a control rod 310 is releasably coupled to an end of the implant 308. The control rod 310 may also be made of a flexible and biocompatible material, such as nitinol.

FIG. 3B is perspective view of a distal end of the cannula 210 of FIG. 3A, illustrating additional details that may be associated with some embodiments. In the example of FIG. 3B, the conduit 306 is slidably disposed within the cannula 210 and can be advanced partially beyond the distal opening 212 of the cannula 210. A distal opening 312 of the conduit 306 is shown extending from the distal opening 212 of the cannula 210 in the example of FIG. 3B. The conduit 306 can have a sufficient length to enable it to extend beyond the distal opening 212 of the cannula 210 at least half-way around Schlemm’s canal. In some embodiments, the length may be sufficient to extend fully around Schlemm’s canal. For example, a length of about of 16-22 millimeters may be particularly suitable for some embodiments. While the illustrative example of FIG. 3B includes only the distal opening 312, other embodiments of the conduit 306 may include additional openings, such as openings along a side of the conduit 306.

FIG. 3C is another schematic view of a distal end of the cannula 210 of FIG. 3A, illustrating additional details that may be associated with some embodiments. In the example of FIG. 3C, the implant 308 is slidably disposed within the cannula 210 and is advanced partially beyond the distal opening 212 of the cannula 210.

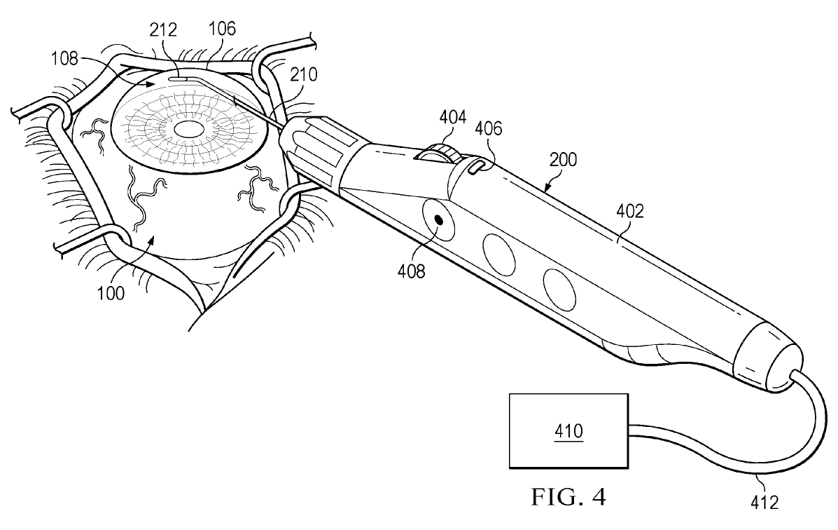

FIG. 4 is a schematic view of another example of the apparatus 200, illustrating additional details that may be associated with some embodiments. For example, the cannula 210 of FIG. 4 extends from a handle 402 and through the cornea 106 of the eye 100. A distal portion of the cannula 210 may be disposed inside the anterior chamber 108 defined by the cornea 106 of the eye 100. In the example of FIG. 4, the cannula 210 may be configured so that the distal opening 212 of cannula 210 can be placed in fluid communication with Schlemm’s canal 118 (not visible in FIG. 4).

The apparatus 200 of FIG. 4 includes a controller 404, an actuator 406, and a switch 408. In some examples, the controller 404 may be a wheel, dial, or similar device, and the actuator 406, the switch 408, or both may be a lever, button, or similar device. In some embodiments, the apparatus 200 may include or can be fluidly coupled to a source of viscoelastic material, such as a visco module 410. A conduit 412 may couple the apparatus 200 to the visco module 410 in some embodiments.

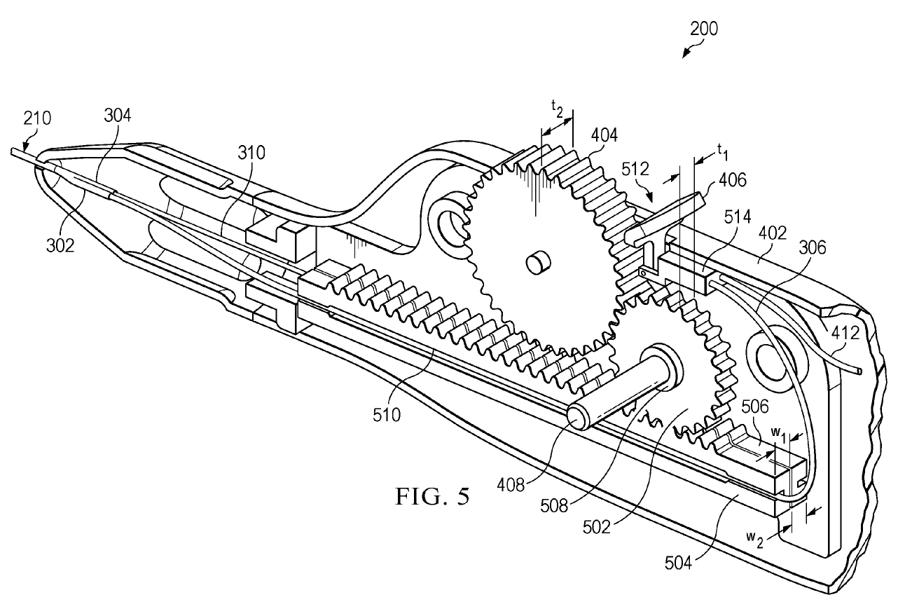

FIG. 5 is a partial section view of the apparatus 200 of FIG. 4, illustrating additional details that may be associated with some embodiments. For example, the controller 404 of FIG. 5 is a gearwheel and part of a gear train mounted within the handle 402. The gear train may include a pinion 502, a conduit rack 504, and an implant rack 506. The conduit rack 504 and the implant rack 506 may be disposed adjacent to and parallel to each other and be configured to move independently of each other. In some examples, the conduit rack 504 may be aligned with the conduit chamber 302, and implant rack 506 may be aligned with the implant chamber 304.

In general, the controller 404 of FIG. 5 is meshed with the pinion 502. The pinion 502 of FIG. 5 has a thickness t1, the controller 404 has a thickness t2, the conduit rack 504 has a width w1, and the implant rack 506 has a width w2. In some examples, the thickness t1 of the pinion 502 may be less than either of the width w1 of the conduit rack 504 and the width w2 of the implant rack 506. The width w1 and the width w2 may be equal in some examples. The thickness t2 of the controller 404 is generally greater than the thickness t1 of the pinion 502. In some embodiments, the thickness t2 may be substantially equal to the combined width of w1 and w2.

As shown in the example of FIG. 5, the switch 408 may comprise or be coupled to a shaft 508, and the pinion 502 may be coupled to the shaft 508. In some examples, the shaft 508 may be slidably mounted to the handle 402 so that actuating the switch 408 moves the shaft 508 and the pinion 502 from a first position to a second position relative to the conduit rack 504 and the implant rack 506. For example, in a first position, the pinion 502 may be meshed with the conduit rack 504, as illustrated in FIG. 5. In a second position (not shown), the pinion 502 may be meshed with the implant rack 506. The thickness t1 of the pinion 502 in the example of FIG. 5 is less than either of the width w1 of the conduit rack 504 and the width w2 of the implant rack 506 so that the pinion 502 is coupled only with the controller 404 and the conduit rack 504 in the first position and only with controller 404 and the implant rack 506 in the second position. The pinion 502 can remain coupled to the controller 404 as the pinion 502 is moved from the first position to the second position.

In the example of FIG. 5, the controller 404 is configured to selectively advance and/or retract either of the conduit rack 504 or the implant rack 506, relative to the cannula 210, depending on the position of the pinion 502. For example, the controller 404 can be rotated in a first direction, which in turn can rotate the pinion 502 in an opposite direction. If the switch 408 is in the first position, such as illustrated in FIG. 5, the pinion 502 is engaged with the conduit rack 504, and rotating the pinion 502 moves the conduit rack 504 linearly in a first direction. Rotating the controller 404 in a second direction, opposite to the first direction, can reverse the rotation of the pinion 502 and move the conduit rack 504 linearly in a second direction, opposite to the first direction. Actuating the switch 408 to move the pinion 502 to the second position, substantially as described above, can disengage the pinion 502 from the conduit rack 504 and engage the pinion 502 with the implant rack 506. If the switch 408 is in the second position, the pinion 502 is engaged with the implant rack 506, and rotating the pinion 502 moves the implant rack 506 linearly in a first direction. Rotating the controller 404 in a second direction, opposite to the first direction, can reverse the rotation of the pinion 502 and move the implant rack 506 linearly in a second direction, opposite to the first direction.

In some examples, the conduit rack 504 may be coupled to the conduit 306. For example, the conduit 306 of FIG. 5 is disposed within a slot 510 of the conduit rack 504. In some examples, an interference fit may hold the conduit 306 in the slot 510. The implant rack 506 may be coupled to the control rod 310. Operating the controller 404 to selectively move the conduit rack 504 and the implant rack 506 substantially as described above can in turn selectively move the conduit 306 and the control rod 310, respectively.

In some embodiments, the actuator 406 may be coupled to a flow controller 512, as illustrated in the example of FIG. 5. The flow controller 512 may be configured for metering of viscoelastic material from visco module 410 (not shown in FIG. 5). As shown in FIG. 5, the actuator 406 may be operatively coupled to a housing 514. In some embodiments, the housing 514 may be enclosed within the handle 402, and the actuator 406 may protrude through an opening in the handle 402, as shown in the example of FIG. 5. The flow controller 512 may be coupled to the conduit 306 and to the conduit 412.

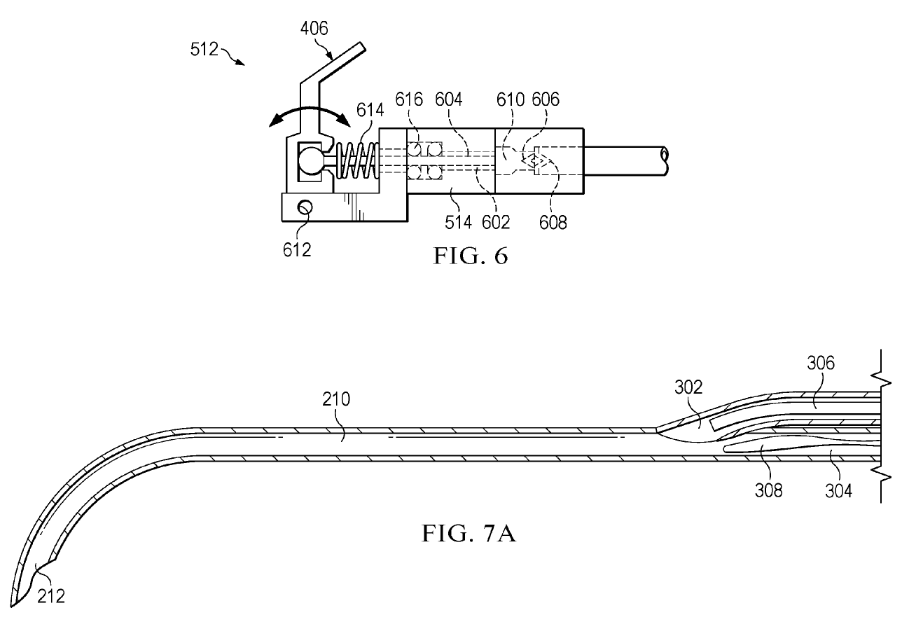

FIG. 6 is a schematic side view of the flow controller 512 of FIG. 5, illustrating additional details that may be associated with some embodiments. For example, the housing 514 of FIG. 6 comprises a cylinder 602. The flow controller 512 of FIG. 6 also comprises a plunger 604, an inlet check valve 606, an outlet check valve 608, and a reservoir 610 fluidly coupled to the cylinder 602, the inlet check valve 606, and the outlet check valve 608. In some embodiments, the inlet check valve 606 and the outlet check valve 608 may be duckbill valves, as illustrated in FIG. 6. The plunger 604 of FIG. 6 may have at least a portion operable to move within the cylinder 602. A pivot pin 612 may couple the actuator 406 to the housing 514, and the actuator 406 may be coupled to the plunger 604 and a return spring 614. Seals 616 between the plunger 604 and the cylinder 602 can provide a barrier to prevent transfer of fluid or viscoelastic material from the reservoir 610 through the cylinder 602, while allowing the plunger 604 to move. In some examples, the seals 616 may be ring seals made of silicone.

In operation, the apparatus 200 can selectively deliver the conduit 306, viscoelastic, and the implant 308 into Schlemm’s canal.

For example, with the pinion 502 in the first position as illustrated in FIG. 5, the controller 404 can move the conduit rack 504 in the first direction to advance the conduit 306 from the conduit chamber 302 through the distal opening 212 into Schlemm’s canal if the distal opening 212 is in fluid communication with Schlemm’s canal. Furthermore, the controller 404 can also move the conduit rack 504 in the second direction to retract the conduit 306 from Schlemm’s canal, through the distal opening 212, and into the conduit chamber 302.

The visco module 410 (shown in FIG. 4) may maintain viscoelastic material at a priming pressure. In a priming configuration, the priming pressure is sufficient to open the inlet check valve 606, allowing the viscoelastic material to move from the visco module 410 through the conduit 412 into the reservoir 610. The actuator 406 can be configured to move the plunger 604 in a first direction to increase the pressure in the reservoir 610 from a first pressure to a second pressure, which can close the inlet check valve 606 and open the outlet check valve 608 to displace the fluid or viscoelastic material from the reservoir 610 through the conduit 306 and into Schlemm’s canal. For example, the actuator 406 of FIG. 6 can move the plunger 604 toward the outlet check valve 608 to increase the pressure in the reservoir 610 from a priming pressure to a delivery pressure. The return spring 614 can return the actuator 406 and the plunger 604 to the initial positions, which can reduce the pressure in the reservoir 610. The diameter and travel distance of the plunger 604 determines the volume of material that can be delivered by each movement of the actuator 406. In other examples, the source of fluid or viscoelastic material may not be pressurized, or sufficiently pressurized, and the actuator 406 can move the plunger 604 in another direction to decrease the pressure in the reservoir 610, which can cause fluid or viscoelastic material to flow into the reservoir 610 through the inlet check valve 606. The controller 404 can enable the conduit 306 to be moved within Schlemm’s canal without administering any viscoelastic material from the conduit 306. Likewise, the actuator 406 can allow viscoelastic material to be administered from the conduit 306 into Schlemm’s canal without moving the conduit 306.

With the pinion 502 in the second position, the controller 404 can move the implant rack 506 in the first direction to advance the control rod 310, which in turn can advance the implant 308 from the implant chamber 304 through the distal opening 212 into Schlemm’s canal if the distal opening 212 is in fluid communication with Schlemm’s canal. Furthermore, the controller 404 can also move the implant rack 506 in the second direction to retract the control rod 310 and the implant 308 from Schlemm’s canal, through the distal opening 212, and into the implant chamber 304.

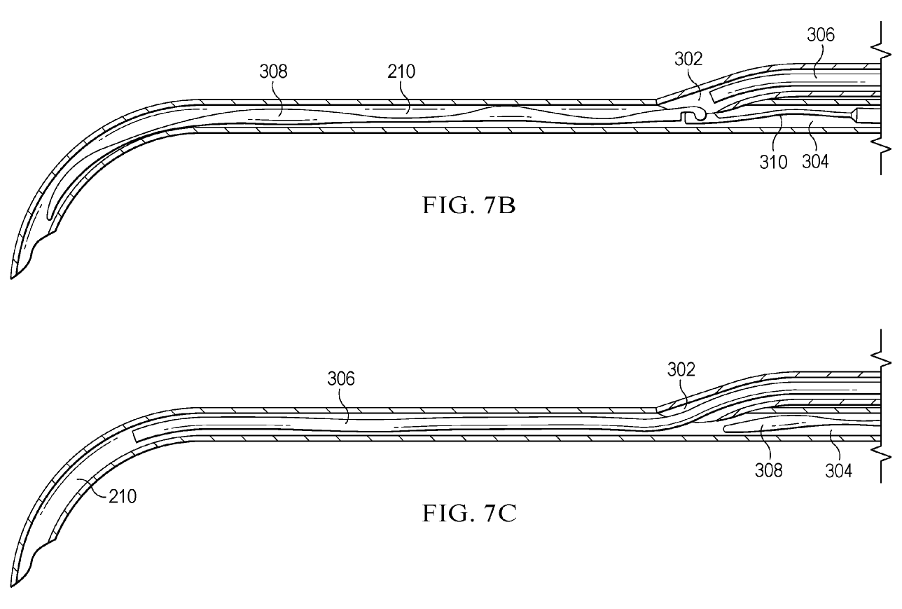

FIGS. 7A-7C are schematic views of another embodiment of the cannula 210 in different configurations. For example, the conduit chamber 302 and the implant chamber 304 of FIGS. 7A-7C are disposed further away from the distal opening 212 than the example of FIG. 3A.

In the configuration of FIG. 7A, the conduit 306 is contained in the conduit chamber 302, and the implant 308 is contained in the implant chamber 304. In the configuration of FIG. 7B, the implant 308 is advanced out of the implant chamber 304 toward the distal opening 212, while the conduit 306 remains contained in the conduit chamber 302. In the configuration of FIG. 7C, the conduit 306 is advanced out of the conduit chamber 302 toward the distal opening 212, while the implant 308 remains contained in the implant chamber 304.

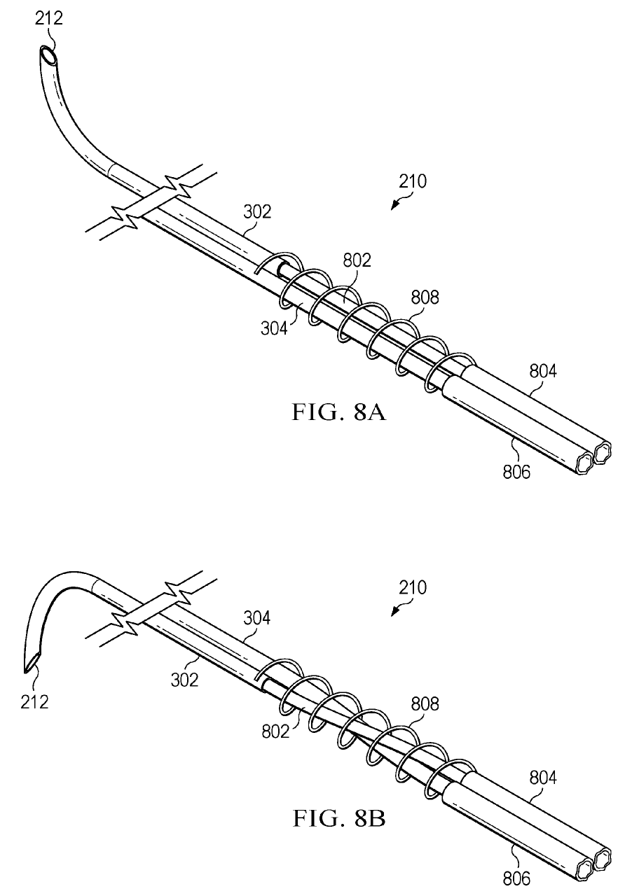

FIGS. 8A-8B are schematic views of another example of the cannula 210, illustrating additional details that may be associated with some embodiments. For example, the conduit chamber 302 of FIGS. 8A-8B may be reinforced with an extrusion 802 to increase column strength and resist kinking. A nesting tube 804 and a nesting tube 806 may hold the conduit 306 (not shown) and the control rod 310 (not shown), respectively, which can move inside of them in a telescoping manner. A spring 808 can surround the conduit chamber 302 and the implant chamber 304 to guide the conduit 306 to the distal opening 212 while substantially reducing or eliminating bowing and kinking. The spring 808 may also be configured to flex so that the conduit chamber 302 and the implant chamber 304 can rotate inside of it if the cannula 210 is rotated.

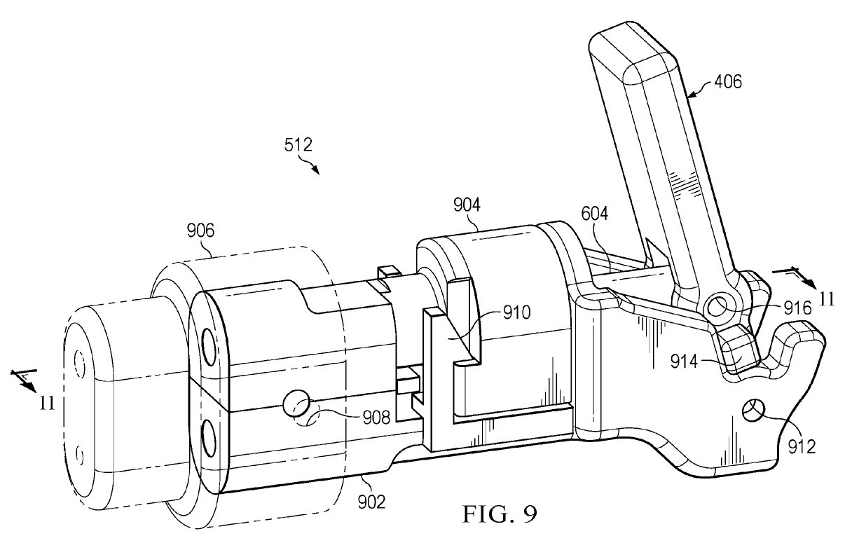

FIG. 9 is a perspective view of another example of the flow controller 512, illustrating additional details that may be associated with some embodiments. For example, the flow controller 512 may comprise a first chassis and a second chassis, such as a lower chassis 902 and an upper chassis 904 illustrated in FIG. 9. The lower chassis 902 can provide support for the actuator 406. An end cap 906 captures both the lower chassis 902 and the upper chassis 904. For example, the end cap 906 of FIG. 9 encloses a portion of the end of each of the lower chassis 902 and the upper chassis 904, which can keep them aligned and adjacent to one another. The end cap 906 may have cross holes 908 through which a dowel or pin (not shown) can be inserted to lock all the chassis components together. A snap hook 910 can provide a locking feature to hold the lower chassis 902 and the upper chassis 904 together. Thus, in the example of FIG. 9, the flow controller 512 may be assembled without adhesives.

A pin (not shown) though hole 912 can couple the actuator 406 to the lower chassis 902. A travel stop block 914 can limit the movement of the actuator 406 between stops. The flow controller 512 may also have a return spring, similar or analogous to the return spring 614 of FIG. 6, which can return the actuator 406 to the original position. A pin (not shown) through a hole 916 can couple the plunger 604 to the actuator 406.

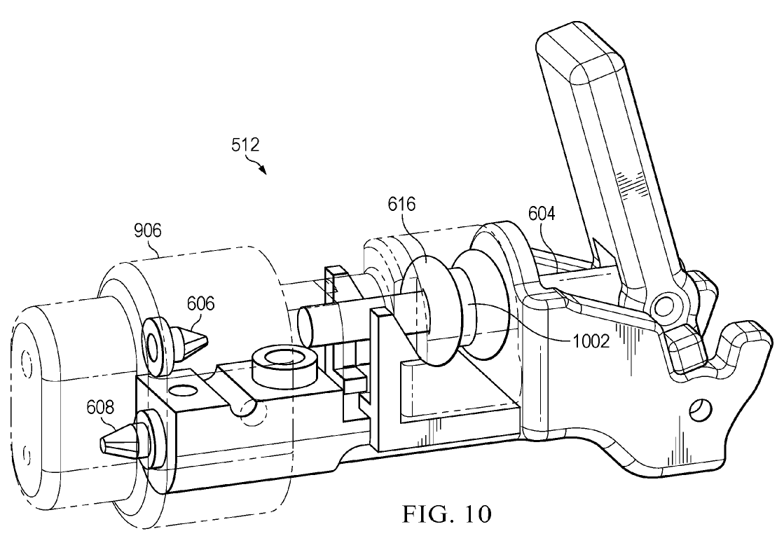

FIG. 10 is another view of the flow controller 512 with the upper chassis 904 removed to illustrate additional details that may be associated with some embodiments. For example, as shown in FIG. 10, the upper chassis 904 may contain the inlet check valve 606, and the lower chassis 902 may contain the outlet check valve 608. In some embodiments, the inlet check valve 606 and the outlet check valve 608 can be captured and sealed by compression of the end cap 906. The plunger 604 can be sealed by the seals 616 with spacer 1002 in between.

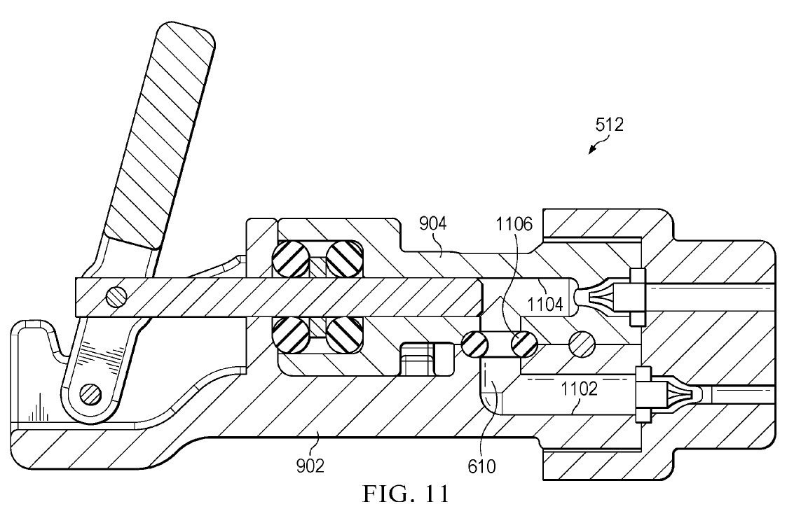

FIG. 11 is a section view of the flow controller 512 of FIG. 9 taken along line 11—11, illustrating additional details that may be associated with some embodiment. As shown in FIG. 11, the lower chassis 902 may contain an outlet flow path 1102, and the upper chassis 904 may contain an inlet flow path 1104. The reservoir 610 may be disposed between, and fluidly couple, the inlet flow path 1104 and the outlet flow path 1102. A ring seal 1106 can provide a seal between upper and lower chassis components.

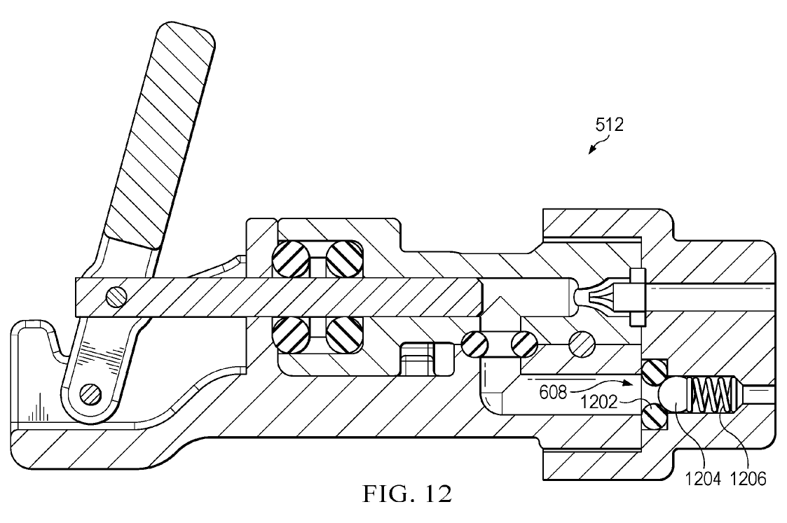

FIG. 12 is a schematic view of another example of the flow controller 512, illustrating additional details that may be associated with some embodiments. For example, the outlet check valve 608 of FIG. 12 comprises or consists essentially of a ball check valve assembly instead of a duckbill valve. The outlet check valve 608 of FIG. 12 generally includes of a ring seal 1202, a ball 1204, and a compression spring 1206. The outlet check valve 608 of FIG. 12 may be advantageous for achieving a higher cracking pressure to prevent unintended flow from a pressurized source of viscoelastic material.

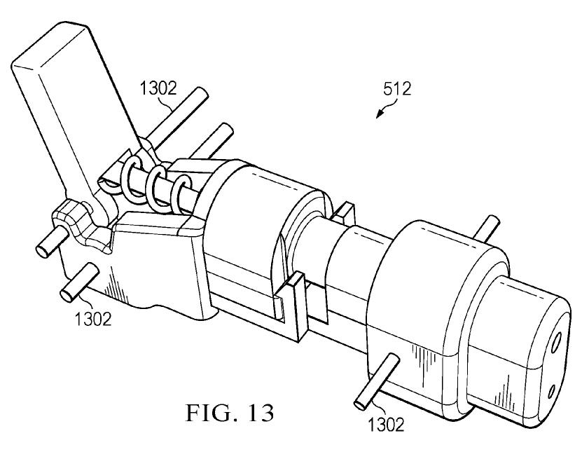

FIG. 13 is a perspective view of another example of the flow controller 512, illustrating additional details that may be associated with some embodiments. For example, the flow controller 512 of FIG. 13 illustrates pins 1302, which can be used to assemble the flow controller 512 without adhesives.

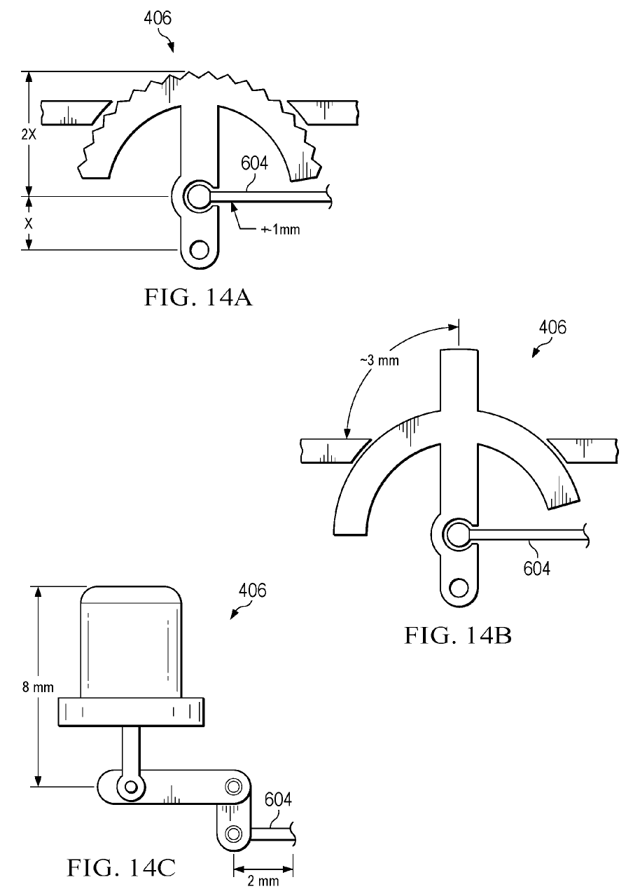

FIGS. 14A-14C are schematic diagrams of other examples of the actuator 406, illustrating additional details that may be associated with some embodiments. For example, the actuator 406 of FIG. 14A is a rolling wheel actuator; FIG. 14B is a lever actuator; and FIG. 14C is a button actuator. Each of these examples can be configured to provide a suitable mechanical advantage for actuating the plunger 604. In some embodiments, for example, a mechanical advantage of 4:1 may be suitable.

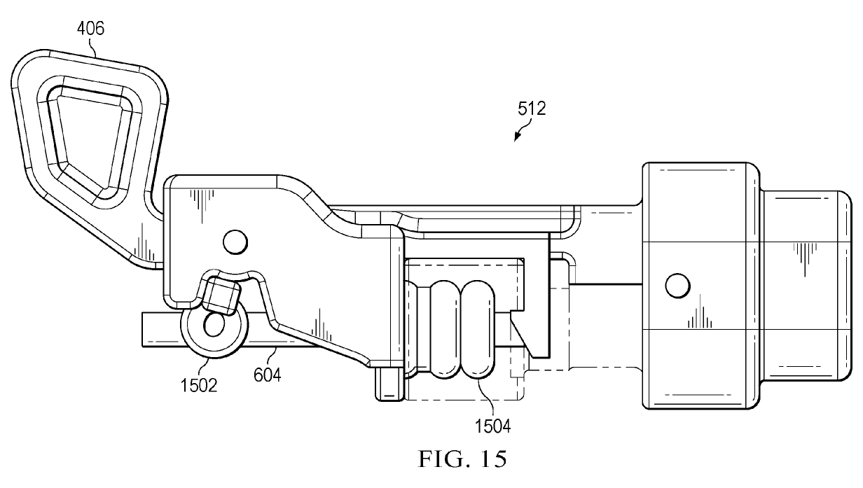

FIG. 15 is a side view of another example of the flow controller 512, illustrating additional details that may be associated with some embodiments. For example, the actuator 406 of FIG. 15 is a button actuator. The button may be coupled to the plunger 604 at an attachment point 1502 to form a bell crank structure with a suitable mechanical advantage. The example of FIG. 15 also illustrates another seal 1504, which may be made from a harder and lubricious material, such as TEFLON, to serve as a wiper seal. In some examples, the seal 1504 can substantially reduce or prevent excess viscoelastic material from reaching the seals 616.

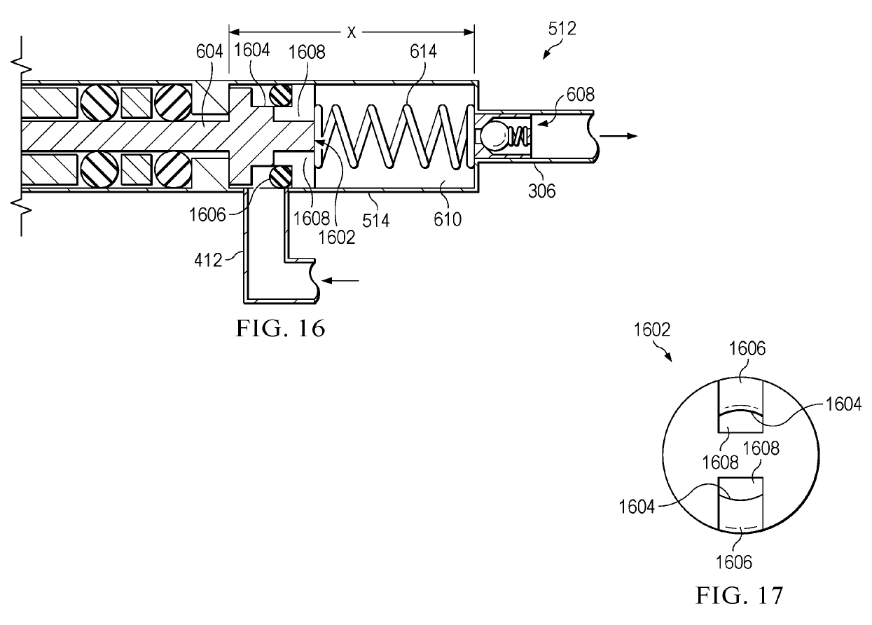

FIG. 16 is a schematic section view of another example of the flow controller 512, illustrating additional details that may be associated with some embodiments in a first configuration. For example, the flow controller 512 may have an inlet, which can be coupled through the conduit 412 to a source of fluid or viscoelastic material, such as the visco module 410 (not shown in FIG. 16). In the example of FIG. 16, the flow controller 512 comprises a piston 1602, which may be coupled to the plunger 604. The return spring 614 can be disposed between the piston 1602 and the housing 514.

The piston 1602 of FIG. 16 comprises a seat 1604. In the example of FIG. 16, the seat 1604 comprises an annular recess, notch, groove, or similar channel around the piston 1602. A piston seal 1606 may be disposed in the seat 1604. A distal end of the piston 1602 of FIG. 16 has a channel 1608, which can fluidly couple the conduit 412 to the reservoir 610. In the configuration of FIG. 16, the channel 1608 is open and the piston seal 1606 can allow transfer of fluid or viscoelastic material from the conduit 412 to the reservoir 610. For example, the visco module 410 can be sufficiently pressurized so that the fluid or viscoelastic material flows around edges of the piston 1602 to the seat 1604, and then between the piston seal 1606 and the piston 1602 into the channel 1608 and to the reservoir 610.

FIG. 17 is a side view of the piston 1602 of FIG. 16, illustrating additional details that may be associated with some embodiments. For example, as shown in FIG. 17, the channel 1608 may form or comprise a recess, notch, or groove in the distal end of the piston 1602, below or inboard of the seat 1604.

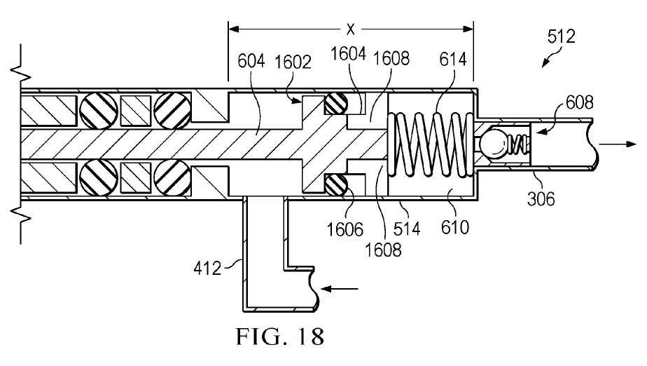

FIG. 18 is a schematic view of the flow controller 512 of FIG. 16 in a second configuration. For example, an actuator, such as the actuator 406 (not shown in FIG. 18), can be configured to move the plunger 604, and thus the piston 1602, from a first position illustrated in FIG. 16 to a second position illustrated in FIG. 18. Additionally, the piston seal 1606 may be configured to slide within the seat 1604 from the first position of FIG. 16, in which the channel 1608 is open, to a second position, as illustrated in FIG. 18, in which the channel 1608 is closed. For example, as the piston 1602 moves from the first position to the second position, friction between the piston seal 1606 and the housing 514 can cause the piston seal 1606 to move relative to the seat 1604. In the closed position of FIG. 18, the piston seal 1606 can provide a barrier to prevent transfer of fluid or viscoelastic material from the conduit 412 to the reservoir 610.

Moving the piston 1602 from the first position to the second position can increase the pressure in the reservoir 610 from a first pressure to a second pressure, which can displace the fluid or viscoelastic material from the reservoir 610 through the check valve 608 to the conduit 306. In the example of FIG. 18, moving the piston 1602 to the second position compresses the return spring 614. If the actuator 406 is released or reversed, the return spring 614 can return the piston 1602 to the initial position, which can reduce the pressure in the reservoir 610 to the first pressure. The diameter and travel distance of the piston 1602 determines the volume of material that can be delivered by each actuation of the actuator 406.

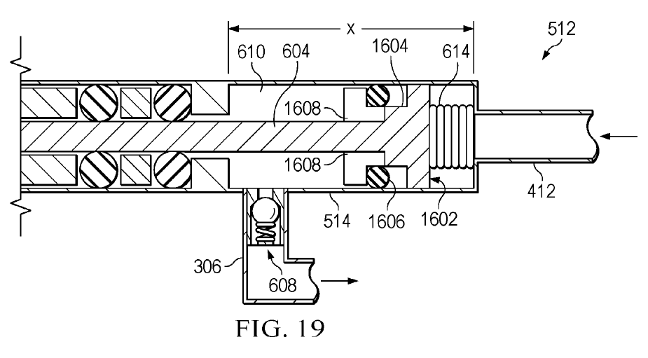

FIG. 19 is a schematic section view of another example of the flow controller 512, illustrating additional details that may be associated with some embodiments. Analogous to the example of FIG. 16, the flow controller 512 of FIG. 19 is shown in a first configuration that allows fluid or viscoelastic material to flow around the piston 1602 to the reservoir 610. For example, the visco module 410 (not shown in FIG. 19) can be sufficiently pressurized so that the fluid or viscoelastic material flows from the conduit 412 around edges of the piston 1602 to the seat 1604, and then between the piston seal 1606 and the channel 1608 to the reservoir 610.

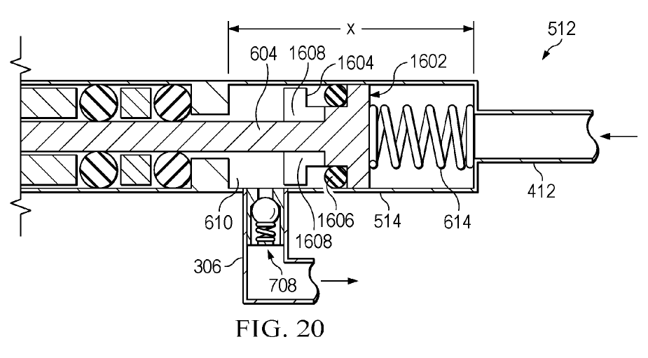

FIG. 20 is a schematic section view of the flow controller 512 of FIG. 19 in a second configuration, analogous to the example of FIG. 18. For example, an actuator, such as the actuator 406 (not shown in FIG. 20), can be configured to move the plunger 604, and thus the piston 1602, from a first position illustrated in FIG. 19 to a second position illustrated in FIG. 20. Additionally, the piston seal 1606 may be configured to slide within the seat 1604 from the first position of FIG. 19 to a second position, as illustrated in FIG. 20. For example, as the piston 1602 moves from the first position to the second position, friction between the piston seal 1606 and the housing 514 can cause the piston seal 1606 to move relative to the seat 1604. In the second position of FIG. 20, the piston seal 1606 can provide a barrier to prevent transfer of fluid or viscoelastic material from the conduit 412 to the reservoir 610.

Moving the piston 1602 from the first position to the second position can increase the pressure in the reservoir 610 from a first pressure to a second pressure, which can displace the fluid or viscoelastic material from the reservoir 610 through the conduit 306. More particularly, in the example of FIG. 20, the return spring 614 is compressed in when the piston 1602 is in the first position. If the actuator 406 is released or reversed, the return spring 614 can move the piston 1602 to the second position, which can increase the pressure in the reservoir 610 to the second pressure. The actuator 406 can be activated or reversed to return the piston 1602 to the first position. The diameter and travel distance of the piston 1602 determines the volume of material that can be delivered by each actuation of the actuator 406.

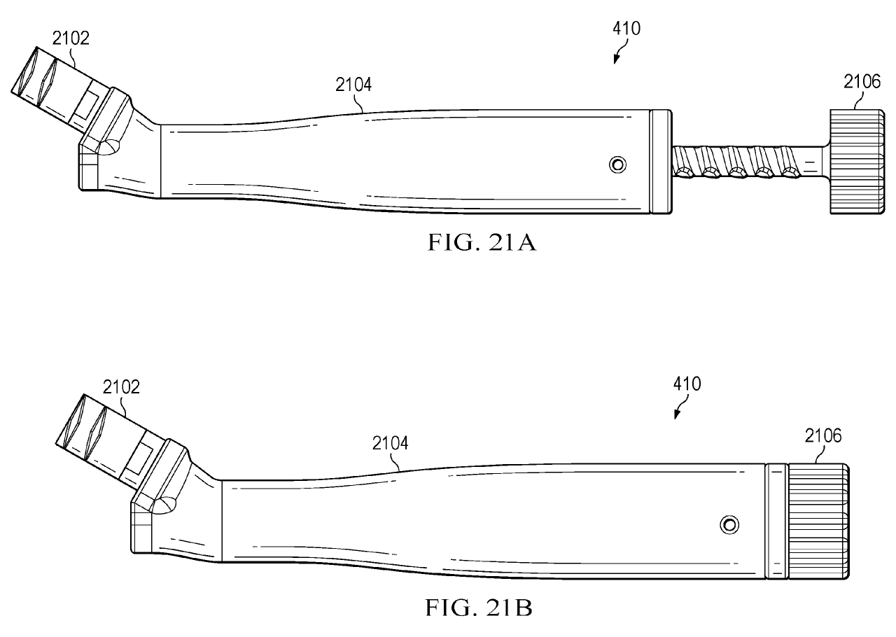

FIGS. 21A and 21B are side views of an example of the visco module 410, illustrating additional details that may be associated with some embodiments. In the example of FIGS. 21A and 21B, a connector port 2102 may be attached to a body 2104. The connector port 2102 can provide an inlet for filling the visco module 410 with visco elastic material. Once filled, a pressure screw 2106 can be tightened, as shown in FIG. 21B.

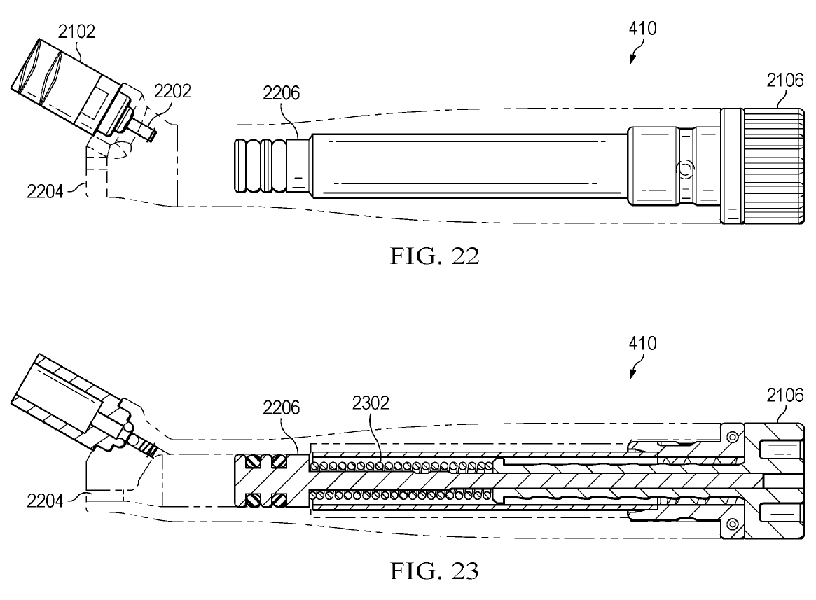

FIG. 22 is a transparent view of the visco module 410 of FIG. 21B, illustrating additional details that may be associated with some embodiments. For example, a check valve 2202 can prevent leakage out of the connector port 2102 in some embodiments. In the example of FIG. 22, the check valve 2202 is a ball check valve. An outlet 2204 can be connected to a delivery apparatus, such as the apparatus 200. The visco module 410 of FIG. 22 also includes a piston 2206, which can be advanced to force viscoelastic material through the outlet 2204.

FIG. 23 is a section view of the visco module 410 of FIG. 22, illustrating additional details that may be associated with some embodiments. For example, the visco module 410 may include a compression spring 2302, which can be compressed to place a force on the piston 2206 if the pressure screw 2106 is tightened.

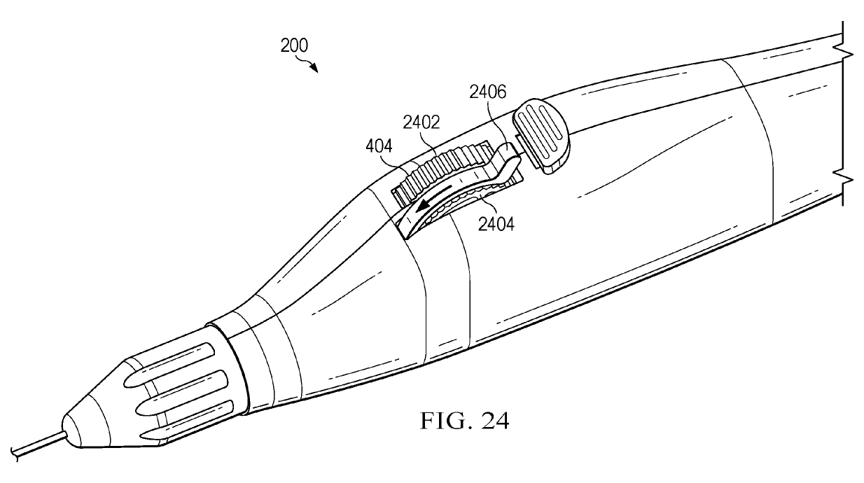

FIG. 24 is a perspective view of another example of the apparatus 200, illustrating additional details that may be associated with some embodiments. For example, the controller 404 of FIG. 24 includes a conduit controller 2402 and an implant controller 2404. In this example configuration, the switch 408 may be eliminated, and the conduit controller 2402 and the implant controller 2404 may independently operate the conduit rack 504 (not shown in FIG. 24) and the implant rack 506 (not shown in FIG. 24), respectively, substantially as described above.

In some embodiments, the apparatus 200 may also have a protective cover 2406, which can be configured to prevent inadvertent movement of the implant controller 2404 while operating the conduit controller 2402, or vice versa. The protective cover 2406 of FIG. 24, for example, may be rotated forward to expose the implant controller 2404 for operation. In some embodiments, rotating the protective cover 2406 may activate an interlock (not shown) to prevent the operation of the conduit controller 2402.

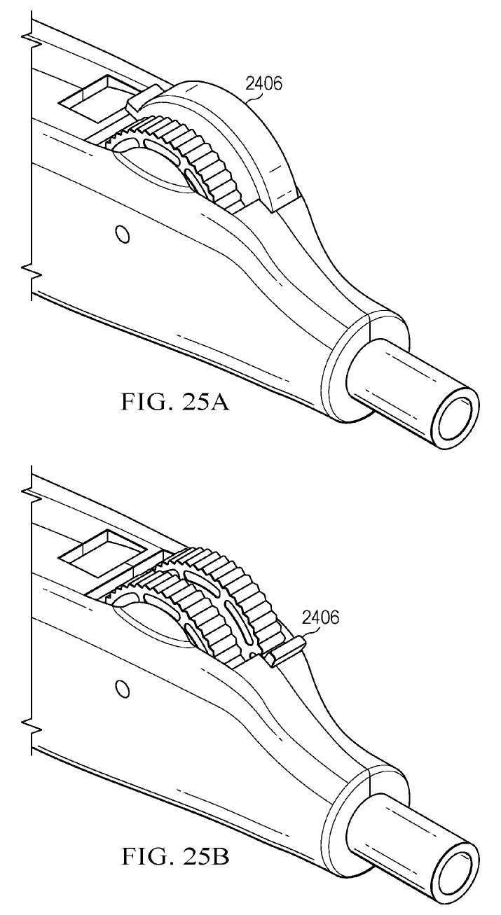

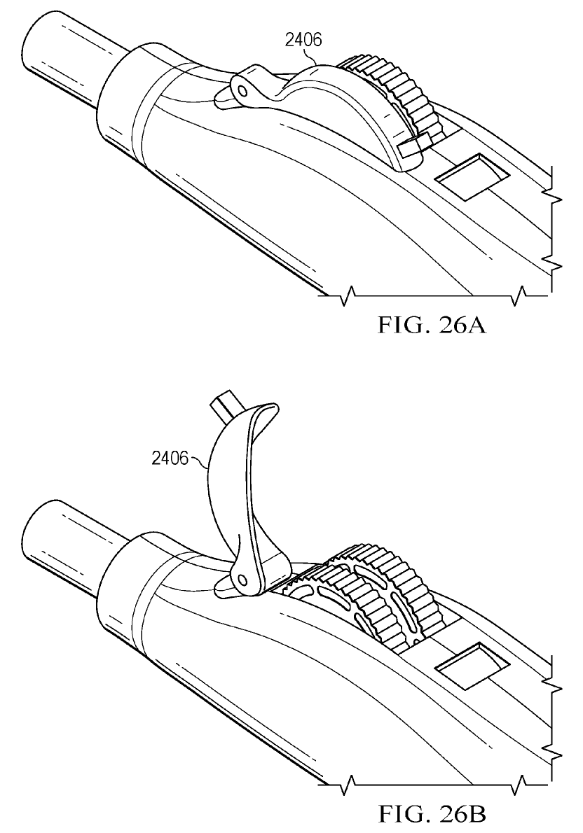

FIGS. 25A-25B and 26A-26B illustrate various embodiments of the protective cover 2406.

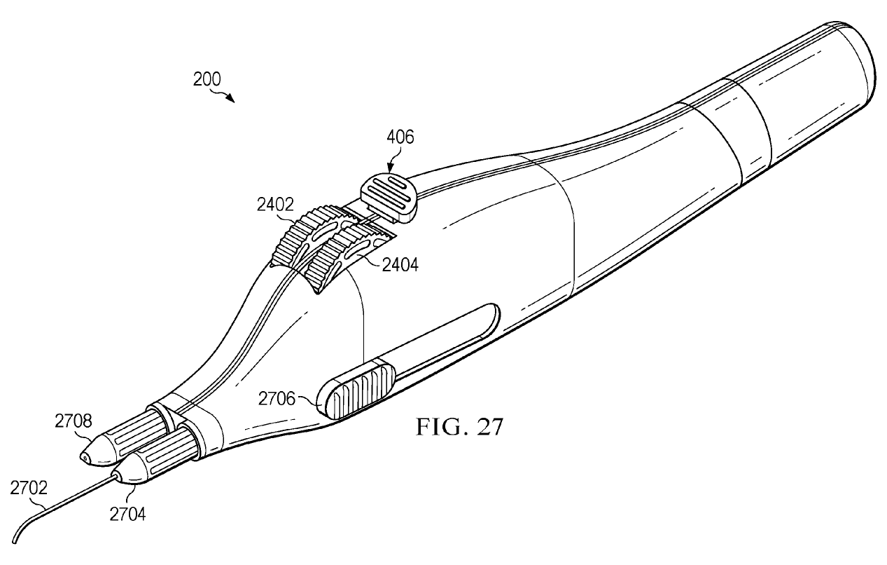

FIG. 27 is a perspective view of another example of the apparatus 200, illustrating additional details that may be associated with some embodiments. Some embodiments of the apparatus 200 may have more than one cannula, which may be extendable and retractable. In the example of FIG. 27, the apparatus 200 is illustrated with an implant cannula 2702 extending from an implant hub 2704. The implant cannula 2702 may be coupled to a slider 2706, which can extend and retract the implant cannula from the implant hub 2704. In some examples, the implant hub 2704 may be rotatable to orient the tip of the implant cannula for an optimal delivery position. A corresponding conduit cannula (not shown in FIG. 27) may be extended and retracted from a conduit hub 2708, which may also be rotatable.

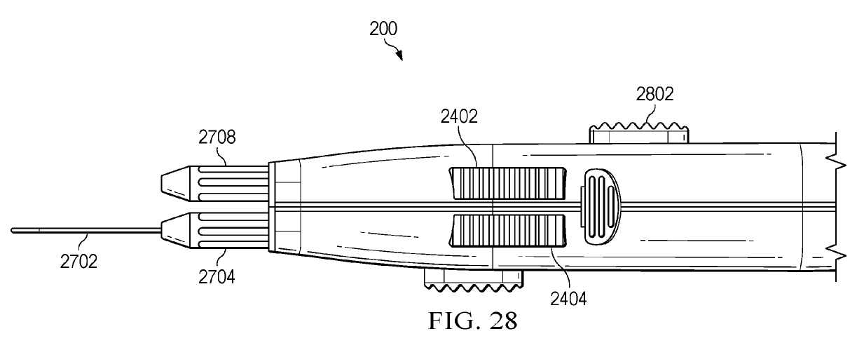

FIG. 28 is a top view of the apparatus 200 of FIG. 27, illustrating additional details that may be associated with some embodiments. For example, the apparatus 200 of FIG. 28 has a slider 2802, which may be configured to extend and retract the conduit cannula (not shown) from the conduit hub 2708.

If the implant cannula 2702 is extended, an implant, such as the implant 308 (not shown), can be deployed through the implant cannula 2702 with a controller, such as the implant controller 2404, substantially as described above. Similarly, if the conduit cannula is extended, a conduit, such as the conduit 306 (not shown), can be deployed through the conduit cannula with a controller, such as the conduit controller 2402, substantially as described above.

The systems, apparatuses, and methods described herein may provide significant advantages. Some embodiments may be particularly advantageous for treating glaucoma with canaloplasty and ocular implants. For example, some embodiments provide a single-delivery form factor for canaloplasty and implants, which can enable more efficient performance of the combination procedure. Additionally, or alternatively, some embodiments can provide a consistent and fixed volume delivery of viscoelastic material for various surgical procedures, including canaloplasty.

While shown in a few illustrative embodiments, a person having ordinary skill in the art will recognize that the systems, apparatuses, and methods described herein are susceptible to various changes and modifications that fall within the scope of the appended claims.

Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use. For example, in some configurations, the flow controller 512 and/or the visco module 410 may be combined or sold separately.

The claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

What is claimed is:

1. An apparatus for delivering viscoelastic material and an implant to an eye, the apparatus comprising:
a cannula comprising a conduit chamber and an implant chamber;
a conduit rack aligned with the conduit chamber;
a conduit coupled to the conduit rack, the conduit configured to move through the conduit chamber and to be coupled to a source of the viscoelastic material;
an implant rack aligned with the implant chamber;
a control rod coupled to the implant rack and configured to be coupled to the implant in the implant chamber; and
a controller configured to selectively move the conduit rack or the implant rack.

2. The apparatus of claim 1, further comprising:
a pinion coupled to the controller; and
a switch operable to move the pinion from a first position to a second position, wherein the pinion is coupled to the conduit rack in the first position and is coupled to the implant rack in the second position;
wherein moving the controller causes the pinion to move the conduit rack relative to the conduit chamber if the pinion is in the first position and causes the pinion to move the implant rack relative to the implant chamber if the pinion is in the second position.

3. The apparatus of claim 2, wherein:
the switch comprises a shaft; and
the pinion is coupled to the shaft.

4. The apparatus of any of claims 2-3, wherein:
the conduit rack has a first width;
the implant rack has a second width; and
the pinion has a thickness that is less than either of the first width and the second width.

5. The apparatus of claim 4, wherein the controller has a thickness that is greater than the thickness of the pinion.

6. The apparatus of any of claims 2-5, wherein the pinion is configured to remain coupled to the controller as the pinion is moved from the first position to the second position.

7. The apparatus of claim 1, wherein the controller comprises a conduit controller coupled to the conduit rack and an implant controller coupled to the implant rack.

8. The apparatus of any preceding claim, wherein the implant chamber is disposed parallel to the conduit chamber.

9. The apparatus of any preceding claim, wherein the implant rack is disposed parallel to the conduit rack.

10. The apparatus of any preceding claim, wherein the conduit chamber and the implant chamber are configured to be rotatable within the cannula.

11. The apparatus of any preceding claim, further comprising:
a flow controller coupled to the conduit and configured to couple the conduit to the source of viscoelastic material; and
an actuator configured to operate the flow controller to deliver viscoelastic material through the conduit to the eye.

12. An apparatus for delivering viscoelastic material and an implant to an eye, the apparatus comprising:
a conduit cannula;
a first slider configured to extend and retract the conduit cannula;
an implant cannula;
a second slider configured to extend and retract the implant cannula;
a conduit rack aligned with the conduit cannula;
a conduit coupled to the conduit rack, the conduit configured to move through the conduit cannula and to be coupled to a source of the viscoelastic material;
an implant rack aligned with the implant cannula;
a control rod coupled to the implant rack and configured to be coupled to the implant; and
a controller configured to selectively move the conduit rack or the implant rack.

13. The apparatus of claim 12, wherein the controller comprises a conduit controller coupled to the conduit rack and an implant controller coupled to the implant rack.

14. The apparatus of claim 12 or claim 13, wherein the implant rack is disposed parallel to the conduit rack.

15. The apparatus of any of claims 12-14, further comprising:
a flow controller coupled to the conduit and configured to couple the conduit to the source of viscoelastic material; and
an actuator configured to operate the flow controller to deliver viscoelastic material through the conduit to the eye.

16. An apparatus for eye surgery, the apparatus comprising:
a cannula comprising a conduit chamber and an implant chamber;
a conduit configured to move through the conduit chamber;
a control rod configured to be coupled to an implant in the implant chamber;
a flow controller coupled to the conduit;
a source of viscoelastic material coupled to the flow controller;
a controller configured to selectively extend and retract the conduit and the control rod; and
an actuator configured to operate the flow controller;
whereby the controller is operable to selectively position the conduit through the conduit chamber into the eye and deliver the implant through the implant chamber into the eye, and the actuator is configured to deliver viscoelastic material through the conduit to the eye.

17. The systems, apparatuses, and methods substantially as described herein.
